(19) United States
(12) Patent Application Publication
(10) Pub. No.: US 2010/0174245 A1
(43) Pub. Date: Jul. 8, 2010

(54) System for pretreating the lumen of a catheter

(76) Inventors: Ward Dean Halverson, Cambridge, MA (US); Ying Jun Du, North Billerica, MA (US)

Correspondence Address: Landiorio Teska & Coleman, 260 Bear Hill Road, Waltham, MA, 02451-1018, US

(21) Appl. No.: 12/319,527

(22) Filed: Jan. 8, 2009

(51) Int. Cl. A61L 29/14; C23C 14/35; C23C 14/34

(52) U.S. Cl. 604/265; 204/298.16; 204/298.15; 204/192.1

(57) ABSTRACT

A method of and system for pretreating the lumen of a catheter or small diameter tubing. A vacuum chamber includes a microwave port and a microwave supply subsystem including a microwave generator, and a circular polarizer produces circularly polarized microwaves propagated into the vacuum chamber via the port at a frequency which produces electron cyclotron resonance. A magnetic coil about the vacuum chamber generates a magnetic field in the vacuum chamber with magnetic field lines co-linear with the propagation direction of the microwaves. A catheter manifold positions at least one catheter in the vacuum chamber and supplies a gas within the catheter lumen to generate a plasma in the lumen for pretreating the same.

### FIELD OF THE INVENTION

This subject invention relates to plasma generation systems and catheter treatment systems and methods.

### BACKGROUND OF THE INVENTION

Catheters for various uses, especially polymer catheters, are often pretreated for a variety of reasons using a variety of methods. For example, polymer based medical catheters are pretreated so that biomedical molecules can better bond with the catheter and/or to increase the wettability of the catheter. Without pretreatment, the polymer surfaces of catheters are too inert or have generally inadequate surface properties for attaching coatings or other surface medication. In general, catheters are pretreated to physically and/or chemically modify them. Plasma is often used in the treatment process.

Given the small interior diameter of a typical catheter, it is difficult to pretreat the lumen of a catheter. See U.S. Pat. No. 5,693,196 incorporated herein which describes the difficulty in forming a plasma in the lumen of small diameter tubing. U.S. Pat. No. 5,486,357 describes treating polymeric surfaces, such as lumens of medical tubing, with a radio frequency generated plasma. The use of a radio frequency generated plasma, however, provides a lesser degree of spatial control for treating a surface and, in addition, a very restricted range of plasma parameters. A RF glow discharge as taught by U.S. Pat. No. 5,914,115 is difficult to maintain within the lumen of a tubular article, especially one having a small diameter. Also, a plasma generated by a RF glow discharge is usually not able to pretreat the lumen of a small diameter polymeric catheter. Microwave chemical vapor deposition also does not work very well for treating small diameter catheters.

The electron cyclotron resonance (ECR) plasma technique has promise over conventional methods, but the ECR process must be applied in the appropriate manner to produce the desired results. Published U.S. Patent Application Serial No. 2002/0172780 by the applicant hereof (incorporated herein by this reference) disclosed an earlier ECR treatment method. The subject invention relates to improvements to an ECR plasma technique for treating the lumen (and optionally the exterior) of a catheter.

### BRIEF SUMMARY OF THE INVENTION

It is therefore an object of this invention to provide a new system for and a method of pretreating the lumen of a catheter.

It is a further object of this invention to provide such a system and method which is able to treat multiple catheters at the same time.

It is a further object of this invention to provide such a system and method able to treat the outside as well as the inside of a catheter.

It is a further object of this invention to provide such a system and method which provide more efficient coupling of the microwave power to initiate and maintain a plasma inside a catheter.

The subject invention results from the realization, in part, that circularly polarized microwaves propagated into magnetic field lines co-linear with the propagation direction of the microwaves result in a more efficient coupling of the microwave power to initiate and maintain a plasma inside a catheter to pretreat the same.

The subject invention, however, in other embodiments, need not achieve all these objectives and the claims hereof should not be limited to structures or methods capable of achieving these objectives.

This subject invention features a system for pretreating the lumen of a catheter or small diameter tubing. One preferred system includes a vacuum chamber including a microwave port, and a microwave supply subsystem with a microwave generator and a circular polarizer producing circularly polarized microwaves propagated into the vacuum chamber via the port at a frequency which produces electron cyclotron resonance. A magnetic coil about the vacuum chamber generates a magnetic field in the vacuum chamber with magnetic field lines co-linear with the propagation direction of the microwaves. A manifold fixture positions at least one catheter in the vacuum chamber and supplies a gas within the catheter lumen to generate a plasma in the lumen for pretreating the same. Typically, if the microwaves are right-handed circularly polarized, the magnetic field is parallel to the microwave propagation direction. If the microwaves are left-handed circularly polarized, the magnetic field is anti-parallel to the microwave propagation direction.

One catheter manifold includes a proximal fixture for one end of the catheter and a distal fixture for the other end of the catheter, the distal fixture connected to a gas outlet conduit. The proximal fixture may include a main gear driving a planetary gear fixed to a proximal connector for rotating the catheter for a more uniform plasma treatment. Typically, there are multiple connectors each rotated via a planetary gear.

The microwave generator may produce microwaves at a frequency between 1 GHz and 30 GHz, preferably a frequency of 2.45 GHz. In one example, the magnetic coil is wound to produce a magnetic field axially decreasing in strength producing a magnetic beach in the vacuum chamber aiding in microwave transmission and heating the plasma. Also, the catheter manifold may include means for rotating the catheter and/or for linearly translating the catheter within the vacuum chamber.

The subject invention also features a method of pretreating a lumen of a polymer catheter so it can be coated with a bioactive material. One method includes generating circularly polarized microwaves at a frequency which produces electron cyclotron resonance and propagating the microwaves to a plasma zone, producing a magnetic field in the plasma zone with magnetic field lines co-linear with the propagation direction of the microwaves, and subjecting the polymer catheter to the plasma zone and introducing a gas in the lumen thereof generating plasma in the lumen which pretreats the same. The method may further include pretreating the outer surface of the polymer catheter.

The method may further include the step of coating the pretreated polymer catheter with a bioactive material and/or an anti-microbial material. Examples of bioactive materials include anti-thrombogenic material such as heparin, antithrombin-III—heparin (ATH), collagen, poly hexamethylene biguanide hydrochloride, adenosine diphosphatase (ADPase), tissue factor pathway inhibitor (TFPI), nitric oxide, polyethylene oxide, and/or tetraethylene glycol ether (tetraglyme), thrombomodulin, pentasaccharides, hirulog, hirudin, PPACK, phosphorl choline, plasminogen activators, peptides, heparin cofactor II, aspirin anhydrides, polyethylene oxide conjugated compounds, and mixtures of the above. Examples of anti-microbial materials include selenium, silver, penicillin, tetracycline, sulfa drugs, quaternary ammonium compounds, and/or octenidine dihydrochloride.

The method may further include the step of coating the pretreated polymer catheter with a monomer base layer for coupling with a bioactive material. Examples of monomer base layers include glycidyl methacylate, acrylic acid, acrylamide, allyl alcohol, allyl amine, methacryl chloride, isocyanate ethyl methacrylate ammonium, fluorohydrocarbons, glycidy acrylate, acryloyl chloride and/or modified albumin. The catheters may be fabricated of various polymers including polyurethane, silicone rubber, polyethylene, polypropylene, polycarbonate, polyester, polystyrene, polymethyl methacrylate, and their co-polymers and/or fluorinated hydrocarbons.

The step of generating circularly polarized microwaves may include employing a magnetron generator with a hybrid polarizer to convert linearly polarized microwaves to circular polarization. The generator operates at a frequency between 1 GHz and 30 GHz, typically 2.45 GHz. One preferred method includes creating a magnetic beach.

The plasma may be formed in a flowing or static gas and the plasma zone is defined by the lumen wall of a catheter. In addition, the plasma may be formed in a flowing or static gas and the plasma zone is defined by the vacuum chamber. In one example, the vacuum chamber is vertically oriented with an external solenoid magnet for producing the magnetic field. The step of introducing a gas may include the use of a differential pumping system connected both to a vacuum chamber and to the catheter lumen to cause plasma formation within the lumen region. The step of introducing a gas may also include introducing a Penning gas mixture into the lumen to assist in the initiation of the plasma. Examples of Penning gas mixtures include mixtures of argon and helium, neon and helium, argon and neon, neon and krypton, and/or argon and krypton.

One preferred method includes inserting a fine metallic wire (e.g., titanium) into the catheter lumen to assist in the initiation of the plasma and/or adding a material such as titanium powder or titanium oxide to the lumen wall to assist in the initiation of the plasma.

One method includes the step of rotating the catheter around its longitudinal axis for a more uniform plasma surface treatment and/or longitudinally translating the catheter for a more uniform external plasma surface treatment. In one example, the microwaves are right-handed circularly polarized and the magnetic field is parallel to the microwave propagation direction. In one example, the microwaves are left-handed circularly polarized and the magnetic field is anti-parallel to the microwave propagation direction.

The subject invention also features a polymer catheter pretreated by the method so described. A polymer catheter is pretreated by coating the same with hydrophilic compounds. Hydrophilic compounds include poly ethylene glycol, hyaluronic acid polysaccharides, polyvinyl alcohol, polyisopropyl allylamide, polyvinyl pyrrolidinone, star polymers and/or dendrimers derived from the same. A polymer catheter is also pretreated by coating the same with hydrophobic compounds. Hydrophobic compounds include fluorocarbons, saturated hydrocarbons, and their self assembled monlayers.

### BRIEF DESCRIPTION OF THE SEVERAL VIEWS OF THE DRAWINGS

Other objects, features and advantages will occur to those skilled in the art from the following description of a preferred embodiment and the accompanying drawings, in which:

FIG. 1 is a highly schematic view showing the primary components associated with an example of a system for and method of pretreating the lumen of a catheter in accordance with the subject invention;

FIG. 2 is a schematic cross-sectional view showing the primary components associated with the vacuum chamber of the system shown in FIG. 1;

FIG. 3 is a highly schematic three-dimensional partially cut away view showing an example of a catheter manifold for use with the system shown in FIG. 1;

FIG. 4 is a schematic partially cut-away view of a metal wire inserted in a catheter to be treated in accordance with the subject invention;

FIG. 5 is a schematic partially cut-away view of a catheter tubing with titanium oxide powder grains residing on the lumen walls in accordance with the subject invention;

FIG. 6 is a graph showing plasma heating with a magnetic beach in accordance with one example of the subject invention;

FIG. 7 is a highly schematic view showing another example of a system for pretreating catheters in accordance with the subject invention;

FIG. 8 is a graph showing water contract-angle versus processing time for the ECR plasma processing method of the subject invention; and

FIG. 9 is a graph showing water contact-angle as a function of post treatment time after ECR plasma processing in accordance with the subject invention.

### DETAILED DESCRIPTION OF THE INVENTION

Aside from the preferred embodiment or embodiments disclosed below, this invention is capable of other embodiments and of being practiced or being carried out in various ways. Thus, it is to be understood that the invention is not limited in its application to the details of construction and the arrangements of components set forth in the following description or illustrated in the drawings. If only one embodiment is described herein, the claims hereof are not to be limited to that embodiment. Moreover, the claims hereof are not to be read restrictively unless there is clear and convincing evidence manifesting a certain exclusion, restriction, or disclaimer.

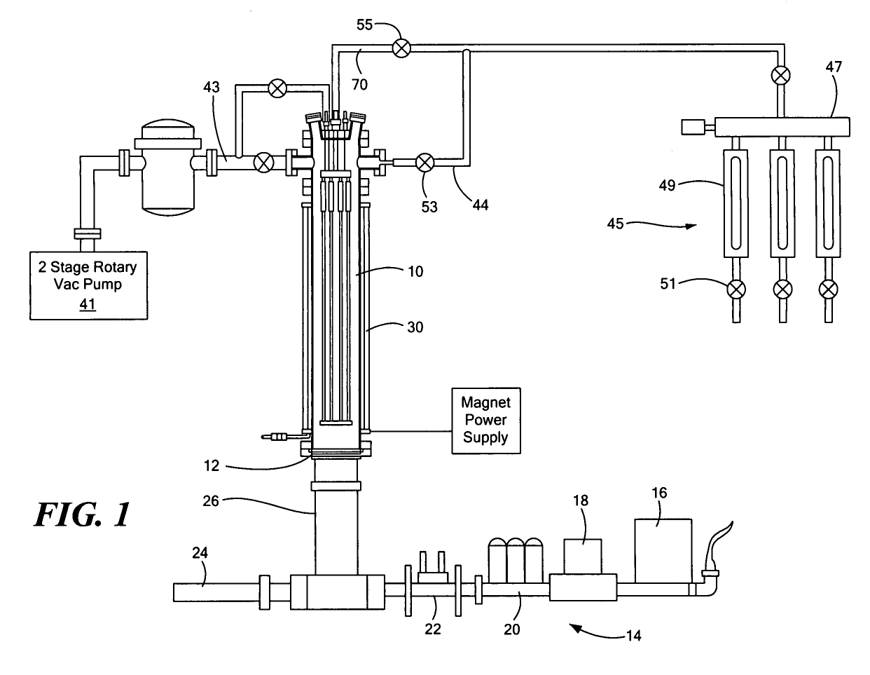

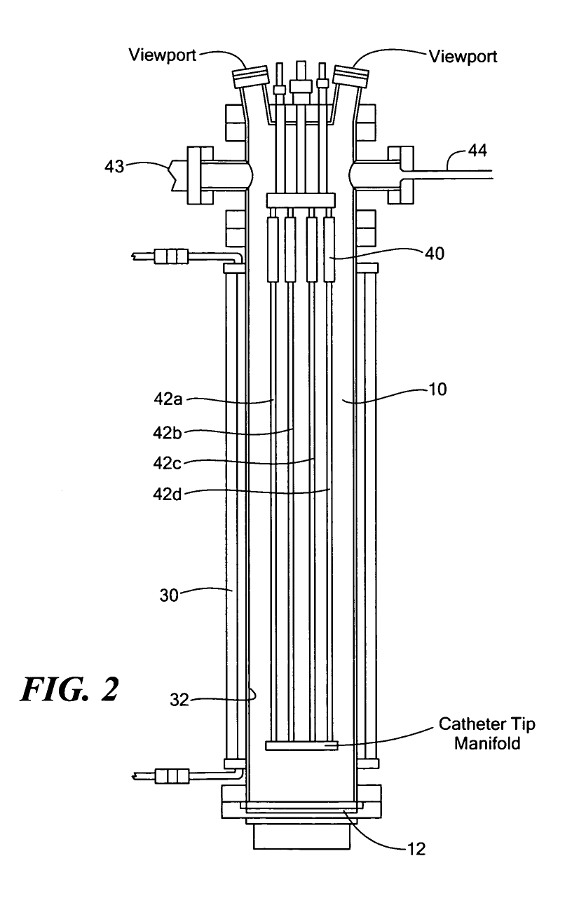

FIG. 1 shows an example of a catheter pretreating system in accordance with the subject invention. Vacuum chamber 10, FIGS. 1-2 (typically a stainless steel vessel) includes microwave port 12 (e.g., a quartz window) and vacuum port 43. Microwave supply subsystem 14 includes inter alia, microwave generator 16, (e.g., a 2.45 GHz magnetron), dummy load 18, three stub tuner 20, directional coupler 22 (e.g., −60 dB), dummy load 24, and circular polarizer 26. Polarizer 26 produces circulary polarized microwaves propagated into vacuum chamber 10 at a frequency which produces electron cyclotron resonance (ECR). In other examples, magnetron 16 or another microwave generator can be operated at between 1 GHz and 30 GHz.

Magnetic coil 30, FIG. 2 about wall 32 of vacuum chamber 10 generates a magnetic field in vacuum chamber 10 with magnetic field lines co-linear with the propagation direction of the microwaves. If the microwaves are right-handed circularly polarized, the magnetic field is parallel to the microwave propagation direction. If the microwaves are left-handed circularly polarized, the magnetic field is anti-parallel to the microwave propagation direction. The unique result is a more efficient coupling of the microwave power to initiate and maintain a plasma than with linearly polarized waves. Although a standard, linearly polarized microwave source can produce electron cyclotron resonance in a magnetized plasma, linearly polarized waves are only one-half as efficient as circularly polarized waves for coupling energy to the plasma.

Catheter manifold 40 positions one or numerous catheters 42a-d and the like within vacuum chamber 10 and supplies a gas or gas mixture within the lumens of the catheters to generate a plasma therein for pretreating the interior of the catheters. Penning mixtures may be used. Other examples include all of the stable noble gases: He, Ne, Ar, Kr, Xe; relatively inert gases (e.g., N₂); reactive diatomic gases (e.g., O₂, Cl₂); more complex molecular gases such as CO₂ or ammonia (NH₃); and organics (e.g., methane (CH₄), allylamines, fluorocarbons). Some of the above gases may also be mixed to enhance the effectiveness of the surface treatment.

Gas inlet 70, FIG. 1 directs gas into the lumens of the catheters within vacuum chamber 10. In such a configuration, the plasma zone is defined by the lumen wall of the catheter. Gas inlet 44 allows gas to be provided into vacuum chamber 10 about the catheters for pretreating the exteriors of the catheters with a plasma. In this configuration, the plasma zone is defined by the vacuum chamber. Gas supply subsystem 45 including manifold 47, flow meters 49, gas metering valves 51, and valves 53 and 55 allow various gasses to be directed into catheter lumen and about the exterior of the catheters (when valve 53 is open). The preferred Penning gas mixture includes mixtures of argon and helium, neon and helium, argon and neon, neon and krypton, and argon and krypton.

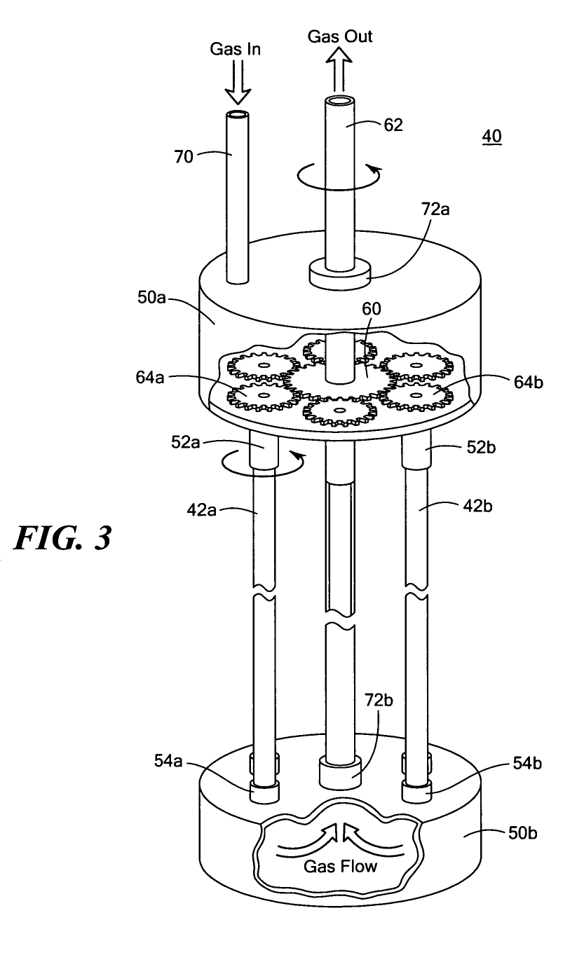

FIG. 3 schematically shows an example of a catheter manifold in accordance with the subject invention including proximal fixture 50a and distal fixture 50b. Included are means to rotate catheters 42a and 42b held by connectors 52a, 52b, respectively, for a more uniform plasma treatment. In this particular example, main gear 60 is driven by shaft 62 rotated by a motor (not shown). Main gear 60 drives planetary gears 64a, 64b, and the like each operatively coupled to connectors 52a, 52b, and the like. Distal fixture 50b includes a similar gear train assembly (not shown for clarity). Gas is supplied to proximal fixture 50a via inlet tube 70. The gas then proceeds through planetary gears 64a, 64b; through connectors 52a, 52b; and into catheters 42a, 42b. The gas then proceeds through connectors 54a, 54b, through the planetary gears associated with distal fixture 50b, and out of the assembly via shaft 62. In this way, the plasma zone is defined by the lumen wall of the catheter.

Rotary vacuum seals 72a and 72b allow rotation of shaft 62 with respect to proximal and distal fixtures 50a, 50b. It may also be desired to linearly translate catheter manifold 40 within vacuum chamber 10, FIG. 2, for example, by including vacuum seals about gas inlet 70 and gas outlet shaft 62 sealing gas inlet 70 and rotating/translating gas outlet shaft 62 with respect to the vacuum chamber.

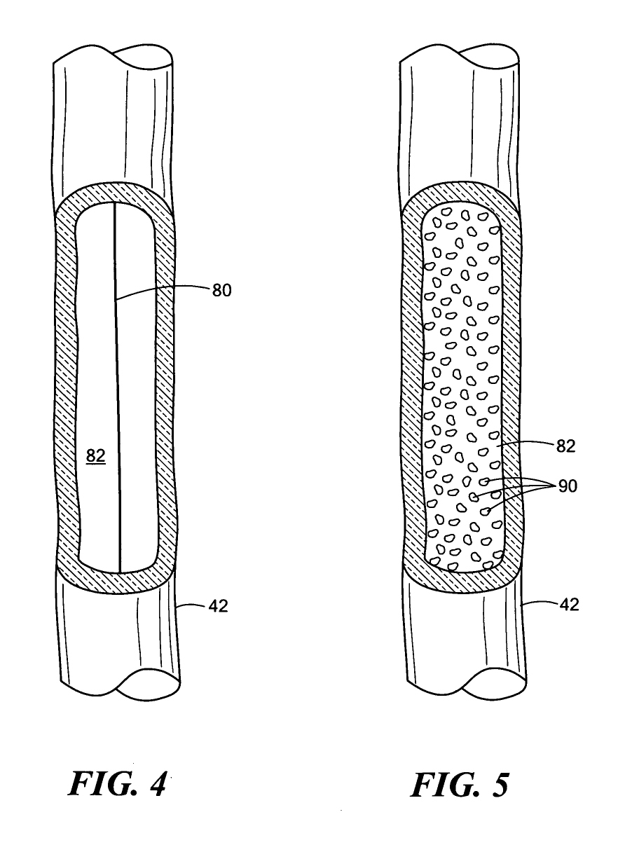

FIG. 4 shows fine titanium wire 80 placed within catheter 42 during plasma treatment of lumen 82. A typical lumen is typically less than 2 mm in diameter. The photochemistry of the titanium aids in the initiation of the plasma within lumen 82. FIG. 5 shows titanium oxide powder particles 92 sprayed onto the walls of catheter 42 lumen 82 before catheter 42 is inserted into the vacuum chamber which will also aid with the initiation of a plasma within lumen 82. It is difficult to establish ECR plasma inside tubing with a small internal diameter (less than 2 mm ID) because the close proximity of the wall induces electron-ion recombination at the solid surface and represents both an “energy drain” for the energetic electrons and a “loss channel” for the ions and electrons. When these losses dominate over ionization of the gas, the plasma cannot be initiated or maintained. This difficulty is even more acute for plasmas formed by other techniques, such as inductively coupled rf plasmas.

Another difficulty in forming plasma in polymer tubing is caused by the presence of an electronically conducting “filler” material in the polymer matrix. The filler (e.g., barium sulfate for radiography) in such “loaded” polymers absorbs energy and attenuates the microwave (or rf) field in the lumen volume, where the plasma is to be generated. Microwave attenuation, when combined with small tubing diameter, produces a serious challenge to plasma generation. The introduction of a fine titanium wire or titanium oxide powder into the tubing lumen has solved these difficulties for ECR plasma generation and maintenance.

It may be desirable to create a “magnetic beach” in vacuum chamber 10, FIG. 2. Accordingly, coil 30 can be wound to produce a magnetic field axially decreasing in strength. The resultant magnetic beach aids in microwave transmission and heating of the plasma. In this way, microwave-plasma coupling is improved. See J. E. Stevens, J. L Cecchi, Y. C. Huang, and R. L. Jarecki, Jr. “Optimized microwave coupling in an electron cyclotron resonance etch tool.” J. Vac. Sci. Techol. A 9, 696-701 (1991); and U.S. Pat. No. 5,111,111 incorporated herein by this reference.

Highly efficient plasma heating can be achieved with microwaves tuned to the fundamental gyration frequency (AKA, “electron cyclotron frequency”) of free electrons in a magnetic field,

fᵥ=f_{c}=(1/2π)(eB/m) (sec⁻¹) (1)

where B is magnetic field strength, and e and m are charge and mass of the electron (mks units). At this frequency, the electric field of the microwaves are at “resonance” with the gyrating electrons and accelerate them very efficiently. The same principle is employed in high-energy cyclotron particle accelerators. For a magnetic field of 8.75×10⁻² Tesla (875 Gauss), the fundamental electron gyrofrequency is 2.45 GHz. In general, when the plasma electron density reaches a high enough level to “cut off” wave propagation at the “plasma frequency,” fₚ, electromagnetic waves are reflected and plasma heating cannot occur. The plasma frequency, numerically, is given by,

fₚ=9(nₑ)^{1/2} (sec)⁻¹ (2)

where nₑ is electron density (m⁻³). Thus, from equation 2, when plasma density reaches approximately 7.4×10¹⁶ m⁻³, microwaves of a frequency of 2.45 GHz cannot propagate.

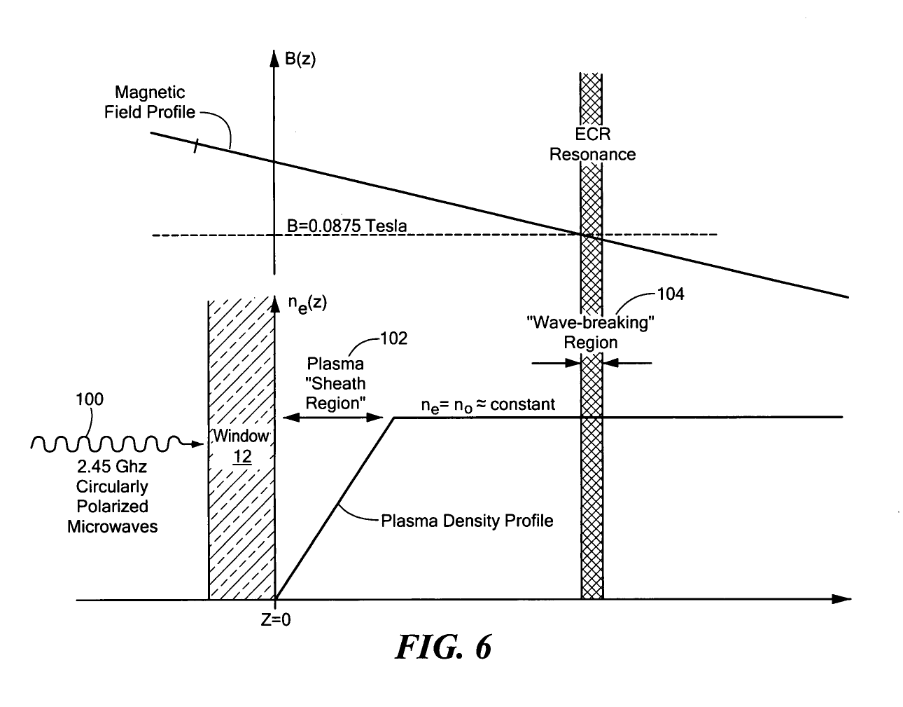

An exception to this general rule is for “right-handed” circularly polarized (rhcp) electromagnetic waves 100, FIG. 6, which can propagate efficiently in the direction of magnetic field lines in “over-dense” plasmas. An example of this type of wave propagation is by low-frequency “whistlers” that are generated by lightning strokes and propagate over extreme distances along the magnetic field lines in the Earth's magnetosphere. Note that “left-handed” circularly polarized waves are cut off at the plasma frequency, unless they are propagating in the anti-parallel direction with respect to the magnetic field.

“Whistler” wave propagation has been shown by the referenced authors to be an effective way to introduce microwaves into a dense plasma and for efficient heating of the plasma. The microwaves are initially launched into the vacuum chamber through window 12 (e.g., quartz) that is transparent at the microwave frequency (e.g., 2.45 GHz). Immediately behind the window, there is a “sheath” region 102 in which un-ionized gas and low density plasma is present. Sheath thickness can vary from a few micrometers to a few millimeters, depending on plasma conditions (principally density and temperature). The plasma density quickly increases with distance to a nearly steady-state value, which can be higher than the “critical” value, with plasma frequency higher than given by equation 2.

To heat an overdense plasma with 2.45 GHz microwaves, Stevens et al. used a magnetic field configuration in which rhcp microwaves were launched into a plasma in which the low-density sheath region was permeated with a longitudinal magnetic field whose strength was greater than the “resonance” value (>875 Gauss). In this region, the “whistler” electromagnetic mode could propagate. By means of an electromagnet with specially wound solenoid coils 30, FIG. 2, the magnetic field strength was made to decrease gradually with longitudinal distance into the plasma, until the resonance condition, equation 1 was reached. Plasma theory shows that as the microwaves travel along the field lines, their propagation speed decreases and amplitude increases until they reach the “resonant surface” defined approximately by equation 1. At this surface, the electromagnetic waves “break” as shown at 104 in FIG. 6 in a similar fashion as water waves on a beach. The “breaking” waves dissipate their energy by non-linear processes and heat the plasma. Heat diffusion and conduction maintains the temperature and ionization density of the plasma in regions not directly heated by the electromagnetic waves. Although the magnetic beach provides very efficient plasma heating, it is not a necessary condition for generating and maintaining an ECR plasma. It has been demonstrated experimentally that the uniform magnetic field of a solenoid coil is sufficient for forming and maintaining plasmas of adequate energy to treat polymer tubing surfaces both internally and externally.

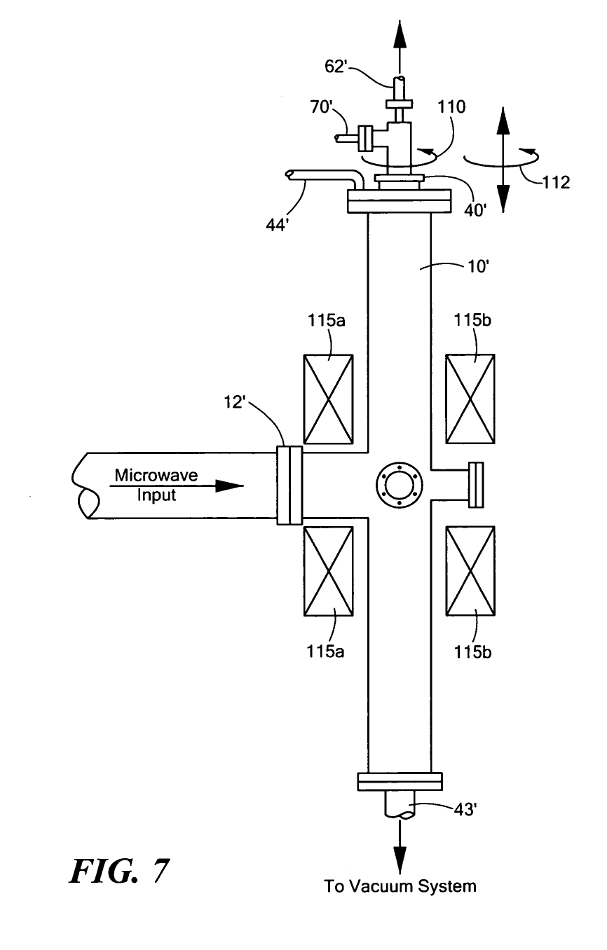

FIG. 7 shows another configuration where vacuum chamber 10′ includes microwave window 12′ and vacuum port 43′. Catheter manifold 40′ includes gas inlet 70′ for distributing gas to the catheter lumens and gas outlet 62′. For treating the exterior of the catheters, gas inlet 44′ is provided. In this example, the catheter manifold 40′ is rotatable as shown by arrow 110 and translatable as shown by arrow 112. Electromagnetic coils 115a and 115b provide the magnetic field lines in the vacuum chamber 10′ co-linear with the propagation direction of the microwaves introduced into chamber 10′ via window 12′.

The inventive ECR plasma system and method allows a reproducible, uniform surface treatment of both flat parts and tubing such as hemodialysis catheters. The latter requires forming ionic plasmas inside the lumen of the small diameter (e.g., 2 mm ID) polymer tubing of the catheter assembly. There are several possible configurations for the catheter processing system, one of which (FIGS. 1-3) has a vertically oriented cylindrical vacuum chamber, around which is wound one or more electromagnet coils that produce an axial magnetic field that can be either axially uniform or designed to generate a magnetic field gradient to form a “magnetic beach” that aids in microwave transmission and heating of the plasma. The catheters under treatment are suspended from a non-metallic fixture 40 which allows both rotary and longitudinal motion; the fixture is constructed of tubing, with manifolds at both proximal and distal ends of the catheters to provide the flow of process gas through the catheters during treatment.

Another design employs a vertical cylinder with a multi-arm “cross” at its center (FIG. 7). Microwaves are input through a window in one “arm” of the cross; electromagnet coils are placed to form a uniform or “magnetic beach” configuration with magnetic field lines parallel to the propagation direction of the microwaves. In this configuration, plasma is generated only in the “cross” region, and the catheters are translated through this zone at a constant speed that determines the “dose” of the surface treatment; other designs are possible.

Uniform surface treatment of external catheter surfaces may require a vacuum-gas feedthrough fixture that physically moves parts within the chamber: both rotational and axial translation. The preferred design is an axially symmetric structure on which more than one catheter or flat part can be mounted. Flat samples or catheters are secured at their upper and lower end and run parallel to the (vertical) axis of the ECR processing chamber. Rotation and translation is incorporated into the design through a planetary gear system that is driven by a central shaft with longitudinal motion capability. Motion control may be accomplished by computer-driven motors.

The fixture for uniform ECR plasma treatment of the interior surface of the catheter tubing includes a manifold 40, FIG. 3 that is independently pumped through “quick connectors” 52 and supplied with flowing gas at the required pressure (typically 1-5×10⁻² Torr) to form an effective ECR process plasma. The subject design allows for simultaneous internal processing of multiple catheters, from three to as many as ten. The fixture system for processing lumens is designed to include rotation and longitudinal motion for processing external surfaces without changing fixtures.

During plasma treatment of catheter lumens, a static gas pressure of a few tens of torr is maintained in the processing vacuum chamber, external to the catheter and fixturing structure. The exterior pressure is high enough (e.g., 200 to 1000 mtorr) to prevent breakdown and plasma formation, which would prevent penetration of the microwaves into the catheter lumens, but low enough to avoid collapse of the catheter walls due to the pressure differential. The pressure differential is established by a gas-handling subsystem 45 and the vacuum subsystem including vacuum port 43 and vacuum pump 41, FIG. 1. Typical catheters are fabricated from relatively soft polymer (e.g., silicone rubber) or from polymer that softens when heated (especially polyurethane, which is used in most hemodialysis catheters). Thus a pressure differential between the interior and exterior walls of the tubing is desirable to prevent tubing collapse before or during plasma processing. Also desirable is a gas pressure in the external volume that is high enough to prevent plasma generation, typically greater than 200 mTorr. A pressure differential of approximately 200 to 1,000 mTorr (0.2 to 1 Torr) is sufficient to prevent both tubing collapse and plasma formation in the volume outside of the tubing.

In some cases, for example catheters constructed of polymer that is “loaded” with a radio-opaque substance such as barium sulfate, it may be difficult to initiate a plasma in the lumen because the “loading” partially absorbs microwave energy. For such cases, it may be necessary to insert material into the lumen to “trigger” plasma formation. One approach is to insert a fine (e.g., 0.1 mm diameter) metal wire 80, such as Ti, Pt, Ag, etc. FIG. 4 on the catheter's axis. The wire provides a source of free electrons when in the microwave field and the free electrons then aid in breakdown and maintenance of the plasma. Another approach is to blow a fine powder of titanium metal or titanium oxide through the lumen before the catheter is mounted on the processing fixture. The powder 90, FIG. 5 that remains on the lumen wall provides a source of free electrons that aid in plasma initiation. Titanium oxide, which forms spontaneously on the surface of titanium metal, is known to be photo-reactive; i.e., it releases electrons when bombarded by ultraviolet or electronic radiation. Following plasma treatment, the wire is removed from the catheter lumen, or the powder is removed by flushing with clean air and/or water.

As shown in FIG. 1, vacuum and gas pressure monitoring gauges are positioned so as to measure pressure in critical locations of the system. Computer controlled pressure gauges are located on the gas-mixing manifold, gas transfer piping, and inside the plasma processing chamber and are equipped with feedback loops to maintain process pressure. A high-vacuum gauge measures the background pressure in the chamber before processing to assure minimal background gas contamination. Gas flow into the processing system is feedback controlled by electronic mass flow controllers that are part of the gas pressure monitoring system.

Monitoring of the plasma is accomplished by plasma emission (PE) spectroscopy, in which the spectrum and intensity of characteristic emission lines are monitored through a window in the processing chamber. The presence, absence, or relative intensity of certain emission lines are sensitive indicators of desired (or anomalous) conditions in the plasma. Additionally, the intensity and time variation of spectrally integrated light emission from the plasma, measured by photodiodes on chamber windows, provide electrical signals that reflect plasma homogeneity, temperature and density, and stability.

In FIGS. 1 and 2, microwaves are injected through a port at the lower end of the vacuum chamber and propagate parallel to the chamber axis and magnetic field lines. When processing the catheter lumen, a pressure differential of a few hundred millitorr is maintained between the exterior and interior surfaces of the catheter tubing such that plasma will form only inside the lumen.

In FIG. 7, microwaves are injected horizontally through the port window at the center of the vacuum chamber and propagate perpendicular to the chamber axis and parallel to magnetic field lines. The magnetic field is provided by electromagnet coils centered on the vacuum chamber “cross.” A fixture similar to that of FIG. 3 provides uniform longitudinal and rotational motion to expose all surfaces of the catheter to the central plasma zone. When processing the catheter lumen, a pressure differential of a few hundred millitorr is maintained between the exterior and interior surfaces of the catheter tubing such that plasma will form only inside the lumen.

### EXAMPLES

Table 1 lists the type and dimensions of samples employed during testing.

TABLE 1
Polymer samples

| Material | Dimensions | Experiments |
|---|---|---|
| Silicone rubber | 6.7 mm OD (20 | External and |
| tubing | Fr*) | internal plasma |
| 70 durometer | 4.0 mm ID | treatment |
| Silicone rubber | 5.3 mm OD (16 | External and |
| tubing 70 durometer | Fr*) | internal plasma |
| | 3.3 mm ID | treatment |
| Silicone rubber flat | 1.91 mm | Plasma treatment |
| MED 4070 70 | thickness | for XPS analysis |
| durometer | | |
| Polyurethane tubing | 3.3 mm OD (10 | External and |
| Pellethane 2363- | Fr*) | internal plasma |
| 55 durometer | 2.7 mm ID | treatment |
| Polyurethane flat | 0.51 mm | Plasma treatment |
| 55 durometer | thickness | for XPS analysis |

*Outside diameter of medical tubing is usually given in Fr (“French”) units.

Tubing samples of approximately 20 cm length were mounted inside a vacuum chamber. The samples were suspended vertically in the center of the chamber and placed so that they were exposed to the plasma in the central ECR zone. The chamber was evacuated to less than 30 mTorr and gas was flowed for 45 minutes or more, under the pressure (70 to 150 mTorr) at which the plasma treatment was to be performed. The low pressure and period of active gas flow removed adsorbed water vapor and residual isopropyl alcohol used for cleaning the exterior tubing surface. The 2.45 GHz microwave supply was then applied at a pre-set power level (usually 100 W), upon which plasma immediately formed. At the end of the process time (0.5 to 15 minutes), microwave power was removed, the vacuum valve closed, and air was admitted into the chamber. The samples were then immediately removed for analysis.

The plasmas were formed in high purity (99.999%) nitrogen, argon, and 50% mixtures of argon and oxygen or nitrogen and oxygen. An oxidizing plasma environment has been shown to produce considerably different surface chemistry on silicone than plasmas of inert or reducing gases such as argon, nitrogen, or ammonia, where the latter tend to create functional silicon-hydride (Si—H) groups onto which bioactive molecules can be bonded. The presence of oxygen in the plasma, on the other hand, causes surface oxidation and the formation of relatively stable C═O groups.

Tubing samples of approximately 40 cm length were mounted inside a vacuum chamber. The tubing lumen was evacuated to less than 30 mTorr and gas was flowed through the tubing for 60 minutes or more, under the pressure (70 to 150 mTorr) at which the plasma treatment was to be performed. The 2.45 GHz microwave supply was then applied at a pre-set power level (usually 100 W) and plasma formed in the tubing. The process time (10 to 120 seconds) was considerably shorter than for exterior treatments, because the plasma was very intense in the small diameter lumen and discolored the polymer if the process was continued for too long a period. After processing, the microwave power was removed, the vacuum valve closed, and air was admitted into the tubing lumen. The samples were then immediately removed for analysis.

The tubing wall was rigid enough to support fill atmospheric pressure; thinner wall tubing would be processed with a partial pressure that is high enough to prevent external breakdown (preventing plasma formation in the lumen) but low enough to avoid collapse of the tubing wall (e.g., 0.2-1 Torr).

Flat sheets of silicone rubber and polyurethane with composition and properties nearly identical to those of the tubing samples were cut into rectangles approximately 2 by 3 cm and suspended in the vacuum chamber by thin strips of the same material. Their placement near the center of the chamber closely resembled that of the tubing samples when treated on their external surface. Procedures for evacuation, gas flow, and plasma treatment were identical to those for the tubing samples that were processed externally.

Tubing exteriors and flat-sample surfaces were evaluated with a goniometer (Kernco, El Paso, Tex.), which is equipped with a movable stage and microscope to measure the tangent angle between small droplets of water and the surface under study. Several contact-angle measurements are averaged to determine the wettability of a given surface. In general, a surface with water contact-angle between 0° and 90° is considered hydrophilic, whereas surfaces with contact angles greater than 90° are considered hydrophobic. Quantitative measurements of surface energy (units: J/m² or dyne/cm) must be performed with a series of different liquids, each with a different intrinsic surface tension; a plot of contact-angle vs. liquid surface tension (at constant temperature) leads to a value of “critical” surface energy that is characteristic of the surface under evaluation. A surface whose critical energy is greater than 71.9 dyne/cm (7.19×10⁻² J/m²) is completely wettable by water (contact angle of 0°). Tabulated critical energy values of clean silicone rubber and polyurethane surfaces are respectively ca. 24 dyne/cm and 35 dyne/cm. Thus, they should have a water contact-angle >0°. As shown below, untreated surfaces of both polymers show water contact-angles near 90°.

### Example 1

### Silicone Rubber Tubing

A series of experiments was performed to investigate surface energy of ECR plasma-treated polymer tubing through water contact-angle measurements. First, the effect of plasma exposure duration was studied on silicone rubber tubing samples of 6.7 mm OD. FIG. 8 summarizes contact-angle data for ECR plasma treatments as a function of process time and gas in the plasma. Other parameters were kept constant: Microwave power, 100 W, total gas flow rate 0.8 liter/min, pressure 80 mTorr. For pure Ar and N₂ plasmas, highest surface energy (lowest contact angle) was found for an exposure time of 8 minutes, while N₂+O₂ treatments produced contact angles in the 100 range down to 30 sec. exposure. The presence of oxygen immediately produced an extremely hydrophilic surface.

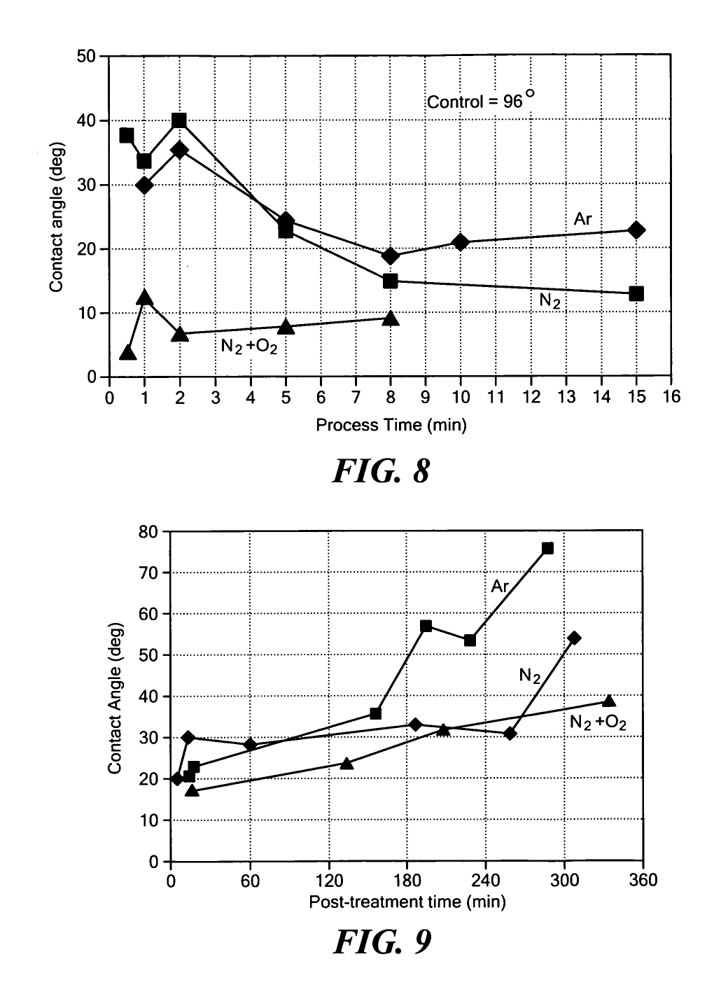

Silicone tubing samples were stored in room air, and water contact-angles were measured as a function of time after ECR plasma processing. FIG. 9 summarizes the data for 8-minute plasma treatments with Ar, N₂ and 50-50% N₂+O₂ plasmas. It will be noticed that the latter plasma produced a higher average initial contact angle than shown in FIG. 8; this sort of experiment-to-experiment variation is typical of the somewhat subjective nature of goniometer measurement of the contact-angle. A similar study of the longevity of a 5-minute N₂ plasma process on the exterior surface of silicone rubber tubing gave data almost identical to that of FIG. 9.

FIG. 9 indicates that the plasma treatment by the inert gas argon does not produce as long-lasting effects as pure nitrogen or nitrogen plus oxygen plasma when the samples are exposed to room air. However, all plasma-processed samples stored in water retained their initial high surface energy for periods of several weeks.

The rise of a liquid in a capillary can be related to surface energy through the equation,

$$h = \frac{2 μ cos θ}{ρ g r} \qquad ( 3 )$$

where μ(dyne/cm) is the difference between surface energy and surface tension of the liquid (dyne/cm), θ is the contact-angle between the liquid and surface, ρ is liquid density (g cm⁻³), g is the acceleration of gravity (981 cm s⁻²), and r is the radius of the capillary (cm). In actual practice, contact-angle θ is nearly impossible to measure accurately, so the cosine term is assumed to be close to unity, a reasonable assumption when h>r. When the rise is small or negative compared to the external liquid level, equation 3 only gives a comparative value for the material's surface energy.

The lumen of silicone rubber tubing of two different diameters (see Table 1) was successfully treated with ECR plasma. Surface activation in the lumen was shown by a dramatic rise in the level of deionized water in the treated samples: after plasma treatment, water in the lumen changed from a convex meniscus below the external water level to a concave meniscus that was well above. Table 2 summarizes data on interior surface treatment of silicone rubber tubing. All plasma treatments produced approximately the same change of surface energy, from hydrophobic to hydrophilic. A control length of silicone tubing that was handled exactly like that of the ECR plasma-treated samples, except without exposure to plasma, showed the same low surface energy as virgin controls.

TABLE 2
ECR plasma treatment of internal (lumen) surface of silicone tubing

| | Tubing | | Water Level | |
|---|---|---|---|---|
| Plasma | ID | Process Time | Rise | |
| Conditions* | (mm) | (min.) | (mm) | Remarks |
| N₂ | 4.0 | 5 | +4.0 | Some |
| | | | | yellowing of |
| | | | | silicone |
| N₂ | 4.0 | 2 | +4.0 | No visible color |
| | | | | change |
| N₂ | 4.0 | 0.5 | +4.0 | No visible color |
| | | | | change |
| N₂ | 4.0 | 0.5 | +7.0 | No visible color |
| | | | | change |
| Ar | 4.0 | 2 | +4.5 | No visible color |
| | | | | change |
| N₂ | 3.3 | 0.5 | +5.0 | No visible color |
| | | | | change |
| Ar | 3.3 | 0.5 | +6.5 | No visible color |
| | | | | change |
| Ar | 3.3 | 0.2 | +6.5 | No visible color |
| | | | | change |
| Control | 4.0 | — | −3.0 | — |
| Control | 3.3 | — | −4.0 | — |

*Microwave power: 100 W. Plasma pressure: 50 mTorr. Gas flow rate: 0.1 liter/min.

The rise of water level by capillary action remained at nearly the same height in plasma-treated silicone samples as long as they were stored in water. Treated samples exposed to room air lost their strong hydrophilicity over a period of 2 to 3 days, although never became as hydrophobic as untreated controls.

### Example 2

### Polyurethane Tubing

Table 3 summarizes experimental conditions for exterior surfaces of polyurethane tubing samples and water contact-angle data immediately after ECR plasma treatment. All plasma gases produced significant reduction of water contact angle on polyurethane tubing, with argon plasma giving the lowest angles.

TABLE 3
ECR plasma treatment of external surface of polyurethane tubing

| | Average Water | Standard |
|---|---|---|
| Plasma | Contact Angle | Deviation |
| Conditions* | (degrees) | (degrees) |
| N₂ | 34 | 5 |
| N₂ | 41 | 7 |
| N₂ + O₂ | 54 | 5 |
| Ar | 29 | 2 |
| Control | 83 | 2 |

*Microwave power: 100 W. Process time: 3 min. Plasma pressure: 70 mTorr. Gas flow rate: 0.5 liter/min.

Surface activation in the lumen of polyurethane tubing was shown by a dramatic change of the level of deionized water in the treated samples. After plasma treatment, water in the lumen changed from a convex meniscus below the external water level to a concave meniscus that was well above. Table 4 summarizes data on interior surface treatment of polyurethane tubing with 3.3 mm OD and 2.7 mm ID. All plasma treatments produced approximately the same change of surface energy, from hydrophobic to hydrophilic. A control length of polyurethane tubing that was handled exactly like that of the ECR plasma-treated samples, except without exposure to plasma, showed the same low surface energy as virgin controls.

TABLE 4
ECR plasma treatment of internal (lumen) surface of polyurethane tubing

| | | Water Level | |
|---|---|---|---|
| Plasma | Process Time | Rise | |
| Conditions* | (sec.) | (mm) | Remarks |
| N₂ | 20 | +6.0 | Tubing partially |
| | | | collapsed |
| Ar | 10 | +6.0 | No visible color |
| | | | change |
| Ar | 20 | +7.0 | No visible color |
| | | | change |
| N₂ | 30 | +5.0 | No visible color |
| | | | change |
| N₂ | 30 | +7.0 | No visible color |
| | | | change |
| Control | — | −5.0 | — |

*Microwave power: 100 W. Plasma pressure: 50 mTorr. Gas flow rate: 0.1 liter/min.

The water level remained at the same height in plasma-treated polyurethane samples as long as they were stored in water. Treated samples exposed to room air lost their strong hydrophilicity over a period of 2 to 3 weeks, although never became as hydrophobic as untreated controls. In general, plasma-treated polyurethane remained hydrophilic considerably longer than silicone rubber under exposure to air.

A polyurethane tubing sample that underwent a 20 sec. argon plasma treatment along with an untreated control was placed in a vessel of toludine-blue dye diluted in water. The dye level in the plasma-treated tubing was about 9 mm above the surface of the liquid and about 12 mm above the meniscus in the untreated tubing. This is a graphic demonstration of the strong capillary effect produced by ECR plasma treatment.

### Example 3

### Monomer Coating

A demonstration was performed on silicone rubber tubing that is nearly identical to that of a hemodialysis catheter. The plasma treatment allowed for the grafting of a monomer-protein coating to the silicone rubber by functionalizing and activating the surface of the silicone polymer.

A 40 cm length of tubing with 5.3 mm OD and 3.3 mm ID (see Table 1) was configured for plasma treatment inside its lumen, and an argon plasma generated in a segment approximately 6 cm in length. Immediately following extinction of the ECR plasma and while still under vacuum, the activated surface of the lumen was exposed to glycidyl methacrylate to form a thin coating on the lumen wall. Upon removal of the silicone tubing from the apparatus, the coated segment was heated to 80° C. for 30 min. to further polymerize the monomer, and then soaked in an albumin solution (0.3 mg/mL) for 18 hrs. For comparison, a control sample of tubing, not treated with argon plasma or monomer, was soaked in albumin.

A visual test determined the presence of the monomer-albumin coating on the plasma-treated tubing versus the untreated control soaked in albumin. The ECR plasma-treated tubing had a translucent appearance, whereas the control was completely transparent, indicating lack of adsorption of the protein. The albumin was bound covalently to the monomer in this experiment and this result can be extrapolated to the application of a heparin-containing or another bioactive substance, in order to achieve a surface with antithrombotic characteristics. The attachment of glycidyl methacrylate and albumin to normally hydrophobic silicone rubber is a proof-of-concept for attaching other bioactive molecules to polymer surfaces. The experiment shows that other gases or low boiling-point liquids, such as NH₃, allylamines, fluorocompounds, and unsaturated hydrocarbons, can also be used for surface activation and functionalization.

In accordance with the subject invention, the right-hand circularly polarized microwaves along a longitudinal magnetic field provide more efficient coupling of the microwave power to initiate and maintain a plasma than with linearly polarized waves. The magnetic beach discussed herein is a further improvement of microwave-plasma coupling. Another feature of the subject invention is the integrated processing chamber and solenoid magnet. Vertical orientation of the processing chamber in a solenoid magnet assists in processing long, thin tubing or catheters. Plasma processing with a differential coupling system as described herein allows treating the inside and the outside of the tubing with different gas plasmas or processed conditions without the need to remove the articles from the chamber or break the vacuum. The use of a “Penning” gas mixture (for example, helium with traces of argon) in a plasma reduces the gas breakdown threshold and aids in initiation and maintenance of a plasma inside very small diameter tubings and/or polymer tubing with conductive “fillers” (for example, barium sulfate). The insertion of the fine metal wire such as Ti along the axis of the very small diameter tubing and/or a polymer tubing with conductive fillers also aids in the initiation of the plasma. Similar results can be obtained by the insertion of a titanium powder on the inside walls of the catheter or tubing. Rotating and/or translating the catheter (including the inserted metal wire) and/or with respect to the processing chamber ensures a uniform surface treatment by the plasma.

One method of pretreating the lumen of a polymer catheter for grafting with a bioactive material in accordance with the subject invention includes generating circularly polarized microwaves at a frequency which produces electron cyclotron resonance and propagating the microwaves to a plasma zone. A magnetic field is produced in the plasma zone with magnetic field lines co-linear with the propagation direction of the microwaves. The polymer catheter is subject to the plasma zone and a gas is introduced in the lumen thereof generating plasma in the lumen which pretreats the same. The system of FIGS. 1-3, FIG. 7, or similar systems may be used to accomplish the method of this invention.

The method may further include pretreating the outer surface of the polymer catheter. After treatment, the pretreated polymer catheter can be coated with a bioactive material (e.g., an anti-microbial material, or an anti-thrombogenic material). Exemplary anti-microbial materials include selenium, silver, penicillin, tetracycline, sulfa drugs, quaternary ammonium compounds, and/or octenidine dihydrochloride. Exemplary anti-thrombogenic materials include heparin, antithrombin-III—heparin (ATH), collagen, poly hexamethylene biguanide hydrochloride, adenosine diphosphatase (ADPase), tissue factor pathway inhibitor (TFPI), nitric oxide, polyethylene oxide, thrombomodulin, pentasaccharide, hirulog, hirudin, PPACK, phosphoral chlorine and/or tetra ethylene glycol ether (tetraglyme). The method may further include the step of coating the pretreated polymer catheter with a monomer base layer for coupling with a bioactive material. Examples of monomer base layer includes glycidyl methacrylate, acrylic acid, acrylamide, allyl alcohol, allyl amine, and/or modified albumin. The method may further include the step of pretreating the catheter with a monomer base layer for coupling with a hydrophilic material such as polyethylene glycol, hyaluronic acid, polysaccharides, polyvinyl alcohol, polyisopropyl acrylamide, polyvinylpyrrolidinone, star polymers and/or dendrimers based on the above. The method may further include the step of pretreating the catheter with a monomer base layer for coupling with a hydrophobic material such as fluorocarbons, saturated hydrocarbons, and their self-assembled monolayers.

Catheters which can be pretreated in accordance with the subject invention include catheters fabricated of polyurethane, silicone rubber, polyethylene, polypropylene, and fluorinated hydrocarbons such as PTFE and Teflon. Preferred polymeric substrates to be treated may include a variety of natural or synthetic polymers. The types of polymers include but not limited to: polyolefins, silicones, acrylic polymers, vinyl halide polymers, polyhydroxyalkanoates (PHAs), polyvinyl ethers, polyvinyl aromatics, polyamides, polyesters, resins, polyurethane, polyimides, polyethers, natural or synthetic rubbers, natural polymers such as wool, cotton, silk, and copolymers consisting two or more polymeric components mentioned above. Examples include but not limited to: acrylonitrile-styrene copolymers, polyacrylate, polyacrylonitrile, polyethylene, polypropylene, polyisobutylene, polydimethylsiloxane, polyetheretherketone (PEEK), polyethersulphone, polymethylmethacrylate, polyethylacrylate, polyvinyl chloride, polytetrafluoroethylene, chlorotrifluoroethylene, fluorinated ethylene-propylene, polyvinylidene fluoride, ethylene tetrafluoroethylene copolymer, perfluoropolyethylene, ethylene propylene copolymer, polyvinyl methyl ether, polyvinylidene fluoride, polyvinylidine chloride, polystyrene, polyvinyl acetate, ethylene-methylmethacrylate copolymers, ABS resins, ethylene-vinyl acetate copolymers, butadiene-styrene copolymers, polyisoprene, synthetic polyisoprene, polybutadiene, butadiene-acrylonitrile copolymers, polychloroprene rubbers, polyisobutylene rubber, ethylene-propylenediene rubber, isobutylene-isoprene copolymers, polyurethane rubber, nylon 66, polycaprolactam, polyethylene teraphthalate, polylactide, polycaprolactone, polyglycolide, polylactide-co-glycolide, polyhydroxybutyrate, phenol-formaldehyde resins, polycarbonates, polyoxymethylenes, polyimides, polyethers, epoxy resins, polyurethanes, wool, cotton, silk, rayon, rayon-triacetate, cellulose acetate, cellulose butyrate, cellulose acetate butyrate, cellulose nitrate, cellulose propionate, cellulose ethers, carboxymethyl cellulose, polyether block amides, polycarbonates, polyvinyl pyrrolidinones, polyvinyl alcohols, styrene acrylonitrile, and composites made from one or more of the above-mentioned polymers and/or fibers or additives. A preferred group of polymers include those of polyurethane, polyolefin, silicone, fluoropolymer, polyester, polyvinyl halide, polyether, polyamide, and blends/copolymers thereof.

A typical processing sequence includes loading one or more previously cleaned tubing segments or catheters onto the processing fixture. The tubing may have a fine metal wire on axis or titanium oxide powder on interior (lumen) walls. Each end of the individual tubes is sealed to the vacuum couplings (“quick disconnects”) on the fixture. The loaded fixture is introduced on the axis of the processing chamber, vacuum-sealed to the chamber, and gas-delivery, vacuum piping, and electrical connections for mechanical movement motors are then attached to feed-through ports in fixture's main vacuum flange.

Air and residual gases are then pumped from the processing chamber, tubing lumens, and all associated piping, gas-handling, gas-distribution, and gas-mixing apparatus.

Process gas(es) for treatment of the lumen wall is flowed at appropriate pressure (1-5×10⁻² Torr) via the processing fixture. Pure gas (e.g., Ar, N₂) is flowed into the processing chamber until a pressure level is reached that will prevent plasma formation external to the tubing lumen (e.g., 0.2 to 1 Torr).

Mechanical rotation or angular oscillation of the processing fixture and/or individual tubing articles is initiated. Longitudinal movement or linear oscillation is initiated, if required. Then the magnetic field is applied at appropriate level. Microwave power is applied at appropriate level for a predetermined processing time. Visual observation and automated diagnostics assure good plasma properties and uniformity. After treatment, microwave power, magnetic field, mechanical movement, and gas flow are turned off.

Following treatment of the tubing lumens, the external walls are treated. The gas previously introduced is pumped from the processing chamber, and a flow of treatment gas(es) is initiated through the chamber, following adjustment to the desired gas species and pressure. Then a plasma is formed in the processing chamber treating the external walls of the tubing. After treatment, gas-delivery and vacuum piping and electrical connections are disconnected from feed-through ports on the main vacuum flange of the fixture. The processing fixture is removed from the chamber and the tubing is unloaded from the fixture. Quality control and inspection of treated articles is performed. Finally, a monomer or polymer attachment layer and/or bioactive coating is applied to the treated articles. The catheter manifold, fittings, and other fixtures may be fabricated of a non-electrically conductive material, such as a high-temperature plastic such as Teflon or Delrin.

Although specific features of the invention are shown in some drawings and not in others, this is for convenience only as each feature may be combined with any or all of the other features in accordance with the invention. The words “including”, “comprising”, “having”, and “with” as used herein are to be interpreted broadly and comprehensively and are not limited to any physical interconnection. Moreover, any embodiments disclosed in the subject application are not to be taken as the only possible embodiments.

In addition, any amendment presented during the prosecution of the patent application for this patent is not a disclaimer of any claim element presented in the application as filed: those skilled in the art cannot reasonably be expected to draft a claim that would literally encompass all possible equivalents, many equivalents will be unforeseeable at the time of the amendment and are beyond a fair interpretation of what is to be surrendered (if anything), the rationale underlying the amendment may bear no more than a tangential relation to many equivalents, and/or there are many other reasons the applicant can not be expected to describe certain insubstantial substitutes for any claim element amended.

Other embodiments will occur to those skilled in the art and are within the following claims.

What is claimed is:

1. A system for pretreating the lumen of a catheter or small diameter tubing, the system comprising:
a vacuum chamber including a microwave port;
a microwave supply subsystem including:
a microwave generator, and
a circular polarizer producing circularly polarized microwaves propagated into the vacuum chamber via the port at a frequency which produces electron cyclotron resonance;
a magnetic coil about the vacuum chamber generating a magnetic field in the vacuum chamber with magnetic field lines co-linear with the propagation direction of the microwaves; and
a catheter manifold positioning at least one catheter in the vacuum chamber and supplying a gas within the catheter lumen to generate a plasma in the lumen for pretreating the same.

2. The system of claim 1 in which the microwaves are right-handed circularly polarized and the magnetic field is parallel to the microwave propagation direction.

3. The system of claim 1 in which the microwaves are left-handed circularly polarized and the magnetic field is anti-parallel to the microwave propagation direction.

4. The system of claim 1 further including a gas inlet into the vacuum chamber supplying gas about the catheter to generate a plasma for pretreating the exterior of the catheter.

5. The system of claim 1 in which the catheter manifold includes a proximal fixture for one end of the catheter and a distal fixture for the other end of the catheter, the distal fixture connected to a gas outlet conduit.

6. The system of claim 5 in which the proximal fixture includes a main gear driving a planetary gear fixed to a proximal connector for rotating the catheter for a more uniform plasma treatment.

7. The system of claim 6 in which there are multiple connectors each rotated via a planetary gear.

8. The system of claim 1 in which the microwave generator produces microwaves at a frequency between 1 GHz and 30 GHz.

9. The system of claim 8 in which the microwave generator produces microwaves at a frequency of 2.45 GHz.

10. The system of claim 1 in which the magnetic coil is wound to produce a magnetic field axially decreasing in strength producing a magnetic beach in the vacuum chamber aiding in microwave transmission and heating the plasma.

11. The system of claim 1 in which the catheter manifold includes means for rotating the catheter around the catheter's longitudinal axis.

12. The system of claim 1 in which the catheter manifold includes means for linearly translating the catheter within the vacuum chamber.

13. A method of pretreating a lumen of a polymer catheter so it can be coated with a bioactive material, the method comprising:
generating circularly polarized microwaves at a frequency which produces electron cyclotron resonance and propagating the microwaves to a plasma zone;
producing a magnetic field in the plasma zone with magnetic field lines co-linear with the propagation direction of the microwaves; and
subjecting the polymer catheter to the plasma zone and introducing a gas in the lumen thereof generating plasma in the lumen which pretreats the same.

14. The method of claim 13 further including pretreating the outer surface of the polymer catheter.

15. The method of claim 13 further including the step of coating the pretreated polymer catheter with a bioactive material.

16. The method of claim 15 in which the bioactive material includes anti-microbial material.

17. The method of claim 15 in which the bioactive material includes anti-thrombogenic material.

18. The method of claim 16 in which the anti-microbial material includes selenium, silver, penicillin, tetracycline, sulfa drugs, quaternary ammonium compounds, octenidine dihydrochloride.

19. The method of claim 17 in which the anti-thrombogenic material includes heparin, antithrombin-III—heparin (ATH), collagen, poly hexamethylene biguanide hydrochloride, adenosine diphosphatase (ADPase), tissue factor pathway inhibitor (TFPI), nitric oxide, polyethylene oxide, tetra ethylene glycol ether (tetraglyme), thrombomodulin, pentasaccharides, hirulog, hirudin, PPACK, phosphorl choline, plasminogen activators, peptides, heparin cofactor II, aspirin anhydrides, polyethylene oxide conjugated compounds, and mixtures of the above.

20. The method of claim 13 further including the step of coating the pretreated polymer catheter with a monomer base layer for coupling with a bioactive material.

21. The method of claim 20 in which the monomer base layer is glycidyl methacylate, acrylic acid, acrylamide, allyl alcohol, allyl amine, methacryl chloride, isocyanate ethyl methacrylate ammonium, fluorohydrocarbons, glycidyl acrylate, acryloyl chloride, and/or modified albumin.

22. The method of claim 13 in which the polymer catheters are fabricated of polyurethane, silicone rubber, polyethylene, polypropylene, polycarbonate, polyester, polystyrene, polymethyl methacrylate, and their co-polymers and/or fluorinated hydrocarbons.

23. The method of claim 13 in which generating circularly polarized microwaves includes employing a magnetron generator with a hybrid polarizer to convert linearly polarized microwaves to circular polarization.

24. The method of claim 22 in which the generator operates at a frequency between 1 GHz and 30 GHz.

25. The method of claim 22 in which the generator operates at a frequency of 2.45 GHz.

26. The method of claim 13 in which producing the magnetic field includes creating a magnetic beach.

27. The method of claim 13 in which a plasma is formed in a flowing or static gas and the plasma zone is defined by the lumen wall of a catheter.

28. The method of claim 13 in which plasma is formed in a flowing or static gas, and the plasma zone is defined by a vacuum chamber.

29. The method of claim 28 in which the vacuum chamber is vertically oriented with an external solenoid magnet for producing the magnetic field.

30. The method of claim 13 in which introducing a gas includes the use of a differential pumping system connected both to a vacuum chamber and to the catheter lumen to cause plasma formation within the lumen region.

31. The method of claim 13 in which introducing a gas includes introducing a Penning gas mixture into the lumen to assist in the initiation of the plasma.

32. The method of claim 31 in which the Penning gas mixture includes mixtures of argon and helium, neon and helium, argon and neon, neon and krypton, and/or argon and krypton.

33. The method of claim 13 further including inserting a fine metallic wire into the catheter lumen to assist in the initiation of the plasma.

34. The method of claim 33 in which the wire material includes titanium.

35. The method of claim 13 further including adding a material to the lumen wall to assist in the initiation of the plasma.

36. The method of claim 35 in which the material is titanium powder.

37. The method of claim 35 in which the material is titanium oxide powder.

38. The method of claim 13 further including the step of rotating the catheter around its longitudinal axis for a more uniform plasma surface treatment.

39. The method of claim 13 further including the step of longitudinally translating the catheter for a more uniform external plasma surface treatment.

40. The method of claim 13 in which the microwaves are right-handed circularly polarized and the magnetic field is parallel to the microwave propagation direction.

41. The method of claim 13 in which the microwaves are left-handed circularly polarized and the magnetic field is anti-parallel to the microwave propagation direction.

42. A polymer catheter pretreated by the method of claim 13.

43. The method of claim 13 further including the step of coating the pretreated polymer catheter with hydrophilic compounds.

44. The method of claim 43 in which hydrophilic compounds include poly ethylene glycol, hyaluronic acid polysaccharides, polyvinyl alcohol, polyisopropyl allylamide, polyvinyl pyrrolidinone, star polymers and/or dendrimers derived from the same.

45. The method of claim 13 further including the step of coating the pretreated polymer catheter with hydrophobic compounds.

46. The method of claim 45 in which hydrophobic compounds include fluorocarbons, saturated hydrocarbons, and their self assembled monolayers.
